(19) United States
(12) Patent Application Publication
(10) Pub. No.: US 2014/0249378 A1
(43) Pub. Date: Sep. 4, 2014

(54) SYSTEMS, METHODS AND KITS FOR MEASURING COUGH AND RESPIRATORY RATE USING AN ACCELEROMETER

(71) Applicant: ISONEA LIMITED, Severna Park, MD (US)

(72) Inventors: Johnny Yat Ming CHAN, Newport Beach, CA (US); Stephen Anthony TUNNELL, Oceanside, CA (US); Joshua Aaron Lawrence JACOBS, Escondido, CA (US)

(73) Assignee: ISONEA LIMITED, Severna Park, MD (US)

(21) Appl. No.: 13/783,257

(22) Filed: Mar. 2, 2013

(51) Int. Cl. A61B 5/08; A61B 5/00

(52) U.S. Cl. CPC A61B 5/0823; A61B 5/0022
USPC 600/301; 600/409

(57) ABSTRACT

The disclosures is directed to cough detectors, methods and kits. Cough detectors can include, for example, a housing adaptable to engage an abdomen or thorax of a mammal; a first sensor comprising an AGM sensor positioned within the housing; a processor in communication with the AGM sensor wherein the processor is capable of receiving a signal from the AGM sensor and analyzing the signal to determine whether the signal is characterizable as a cough; and a power source.

### BACKGROUND OF THE INVENTION

Cough is a common symptom reported to doctors and is a clinical sign of respiratory disease. Often cough is overlooked or weighted less in the physician's mind because of the subjective nature of reporting and when measured nocturnally has been reported to produce a falsely negative cough counts. Physicians have reported that cough count is an important objective sign used by them to both diagnose and treat respiratory disease. Measurement of respiratory data including cough can, for example, be used to evaluate respiratory disease, evaluate a relationship between coughing, other diseases and pathology (such as acid reflux or GERD). Current methods for monitoring cough often have poor accuracy and low patient tolerance. Additionally both nocturnal and daytime measures of cough may vary with nocturnal cough count being suppressed. See. Piirila et al., “Objective Assessment of Cough,” Eur Respir J, 1995, 8, 1949-1956.

Most attempts at cough counting employ either human observation or acoustic means to capture cough rates. Acoustic methods have reported varying success and have been problematic in verifying cough events due to other environmental sounds that approximate the frequency time domain as cough (doors slamming, dog barking, etc). An improved method to validate cough counts can enable physicians to rely on cough measurements to objectively diagnose respiratory disease and assess therapeutic effectiveness.

One mechanism for measuring respiration is disclosed in Lukocius, et al., The Respiration Rate Estimation Method based on the Signal Maximums and Minimums Detection and the Signal Amplitude Evaluation, Electr. And Elect. Engr. 2008, No. 8(88). Other mechanisms include counting coughs from an audio source.

What is needed is: an ambulatory cough counting device that can take measurements electronically; and sufficiently validate counts that is configurable to operate as part of a communication network with a wireless connection.

### SUMMARY OF THE INVENTION

Devices, systems and methods for respiratory rate measurement and/or cough detection in mammals are disclosed. Users and health care practitioners can keep track of measurements from a device, such as those disclosed, by using the networked systems and methods for tracking. A cough detection system using an AGM sensor placed on the abdomen of a subject is provided to detect relative position and cough when the subject is, for example, lying in a prone position. In one aspect, a single sensor is used which is applied using a belt or biocompatible adhesive. The belt is removable and is configured to secure the sensor to the abdomen. In another aspect, a second sensor is provided, such as a second AGM sensor, which is positioned in a second location, such as dorsally. Data acquired by the second sensor is used to cross-correlate against the data acquired by the first sensor to allow for selection and filtration of an optimal axis from the x,y,z plane of the first or second AGM sensor with the strongest amplitude and profile match for measuring coughs. In another configuration, the second sensor is an acoustic sensor placed on the trachea (e.g., a tracheal sensor) which uses either a microphone or PPG (phono pneumo-graph sensor). Data collected from the second acoustic sensor is analyzed along with the data from the first sensor to improve the cough measurement. In still another aspect, two AGM sensors are applied, as described, and an acoustic sensor is provided as a third sensor source. In still other configurations, the acoustic system detects signals passively.

An aspect of the disclosure is directed to a cough detector. Suitable cough detectors comprise: a housing adaptable to engage an abdomen or thorax of a mammal; a first sensor comprising an AGM sensor positioned within the housing; a processor in communication with the AGM sensor wherein the processor is capable of receiving a signal from the AGM sensor and analyzing the signal to determine whether the signal is characterizable as a cough; and a power source. In some configurations, the processor is positioned within the housing and is in wireless or wired communication with the sensor in the housing. The first sensor is also configurable such that it is capable of measuring one or more of each of changes in velocity, movement, orientation of the sensor in a plane, and changes in rotational velocity. The first sensor may also be configurable such that it is capable of determining one or more of a power of a movement resulting from the cough, a motion resulting from the cough, and a change in orientation resulting from the cough. Data relating to the cough can be transmitted to a remote host computer or a data logger via a transmitter. Additionally a suitable securement device capable of securely positioning the sensor adjacent an abdomen, thorax, or trachea of a patient can be provided. The detector can also comprise: one or more secondary sensors, wherein the one or more secondary sensors are selected from the group comprising an AGM sensor, a PPG sensor, and a contact sensor capable of simultaneously sensing and transducing a range of low frequency to high frequency mechanical vibrations, wherein the sensor is configured to be positioned and brought into contact over a lower segment of a cervical trachea of the mammal. The one or more secondary sensors can also be configured such that the one or more secondary sensors are in wireless or wired communication with the first sensor. Additionally, the processor of the secondary sensor can be configured such that it is in communication with the first sensor is in communication with the one or more secondary sensors. Additionally, the processor is capable of comparing one or more signals from the first sensor to one or more signals from the one or more secondary sensors to determine the presence of a cough. One or more processors can be provided which are in communication with the each of the one or more secondary sensors wherein the one or more processors are capable of receiving a signal from the one or more secondary sensors. The one or more processors are capable of comparing one or more signals from the first sensor to one or more signals from the one or more secondary sensors to determine the presence of a cough. A coincidence detector can also be provided. The coincidence detector is configurable for receiving signals from one or more of the first sensor and the one or more secondary sensors, wherein the coincidence detector identifies a coincidence between the signals arriving from two of the sensors and generates a continuous signal for the duration of the coincidence; a duration detector for determining whether the duration of the continuous signal generated by the coincidence detector is within a respective characterization of cough, to thereby identify coughing events; and a register for storing data concerning coughing events identified as such by the duration detector. One or more secondary housings can be provided for the one or more secondary sensors wherein at least one of the one or more secondary housings is adaptable to engage the patient either above or below a diagram in opposition to the first sensor or to contact the patient over a lower segment of a cervical trachea of the mammal. One or more secondary power sources capable of providing power to the one or more secondary sensors. In some configurations, at least one of the one or more secondary sensors is capable of measuring a frequency range of 20 Hz to 2000 Hz. At least one of the one or more secondary sensors can be configured such that it is capable of acoustic sensing using one or more of a vibration sensor and an audible sensor.

Another aspect of the disclosure is directed to a method for detecting and counting coughing events. A suitable method comprises: a) positioning a cough detector comprising a housing, a first sensor comprising an AGM sensor, a power source, and a processor in communication with the AGM sensor on an abdomen or thorax of a mammal; b) detecting one or more of orientation, motion and pressure; c) analyzing the one or more of orientation, motion and pressure sensed to determine whether the detected orientation, motion and pressure corresponds to a movement, motion or pressure resulting from a cough. Additionally, the step of transmitting the detected orientation, motion and pressure to a remote host computer or a data logger can be accomplished via a wired or wireless transmitter. In some configurations, the method also includes a) positioning one or more of a secondary sensor over the abdomen or thorax in opposition to the first AGM sensor or over a lower segment of a cervical trachea for simultaneously sensing and transducing mechanical vibrations emanating from the cervical trachea segment; b) identifying, by a coincidence detector, coincidence of signals; c) determining, by a duration detector, whether the duration of the coincidence is within a range of cough durations to thereby identify coughing events; and d) storing, by a register, data concerning coughing events identified as such by the duration detector. The method can also include the step of sensing one or more of a tracheal vibration or acoustic signal within a frequency range of 20 Hz to 2000 Hz. Additionally one or more signals from the first sensor can be compared to one or more signals from the second sensor to determine the presence of a cough. The first sensor can be removably secured to the abdomen or thorax of the mammal, while the one or more secondary sensors are secured to or more of the abdomen, thorax, or trachea of the mammal.

Still another aspect of the disclosure is directed to a system. Suitable systems comprise: a cough detector configurable to be in communication with a communication network, wherein the cough detector comprises a housing adaptable to engage an abdomen or thorax of a mammal, a first sensor comprising an AGM sensor positioned within the housing; and a processor in communication with the AGM sensor wherein the processor is capable of receiving a signal from the AGM sensor and analyzing the signal to determine whether the signal is characterizable as a cough, and a power source; an alert generator. The alert generator is configurable such that it delivers an alert over the communication network. Additionally, the alert is configurable such that an alert to advise a healthcare provider of a physiological condition as a result of the cough detection. In some configurations a report generator is also provided. Suitable report generators can, for example, compile information received from the sensor and generates a report.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a block diagram showing a representative example of a logic device through which cough and respiratory rate measurement can be achieved;

FIG. 1B is a block diagram of an exemplary computing environment through which cough and respiratory rate measurement can be achieved;

FIG. 1C is an illustrative architectural diagram showing some structure that can be employed by devices through which cough and respiratory rate measurement is achieved;

FIG. 2 is a block diagram showing the cooperation of exemplary components of a system suitable for use in a system where cough and respiratory rate measurement is achieved;

FIG. 3 illustrates a block diagram of a respiratory rate measurement device according to the disclosure;

FIGS. 4A-B illustrate a bottom view and a side cross-sectional view of a respiratory rate measurement device according to the disclosure;

FIGS. 5A-B illustrate potential device placement locations on a torso of a patient;

FIG. 6 is block diagram description of the structural aspects of a secondary or tertiary sensor;

FIG. 7 illustrates cough output for a patient lying on a bed;

FIGS. 8A-F illustrate cough output for a patient when a device is affixed using a tight belt;

FIGS. 9A-F illustrate another example of cough output for a patient when the patient is lying face down on a bed;

FIGS. 10A-B illustrate a cross-correlation of tight belt cough with tight belt data; and

FIGS. 11A-B illustrate a cross-correlation of bed cough data with bed data for different subjects.

### DETAILED DESCRIPTION OF THE INVENTION

### I. Computing Systems

The systems and methods described herein rely on a variety of computer systems, networks and/or digital devices for operation. In order to fully appreciate how the system operates an understanding of suitable computing systems is useful. Aspects of the systems and methods disclosed herein can be enabled as a result of application via a suitable computing system.

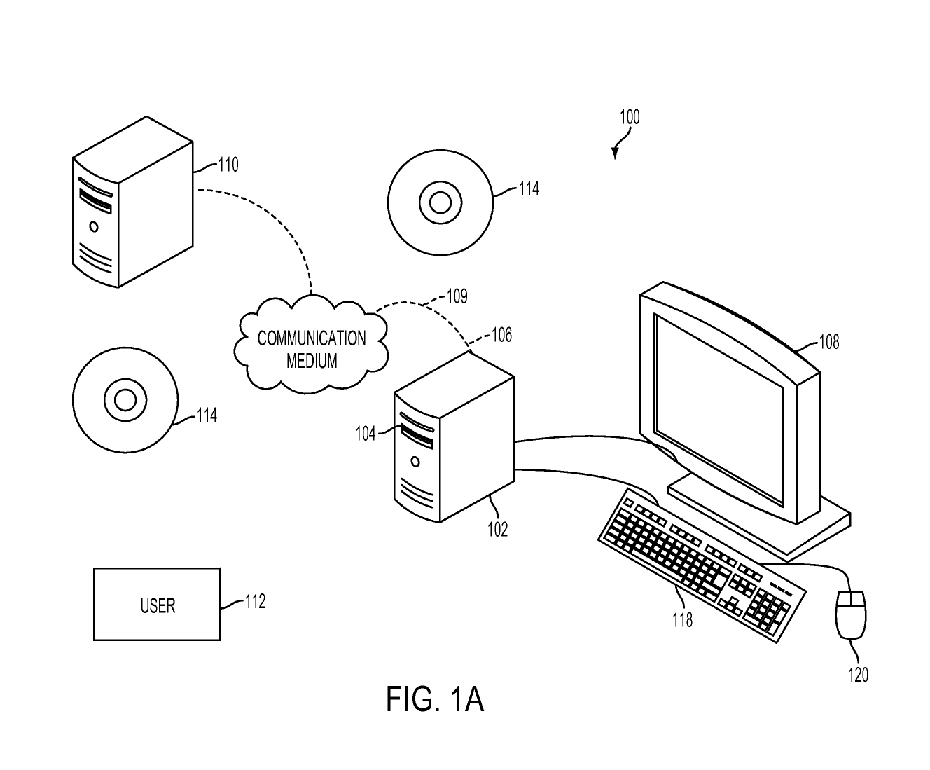

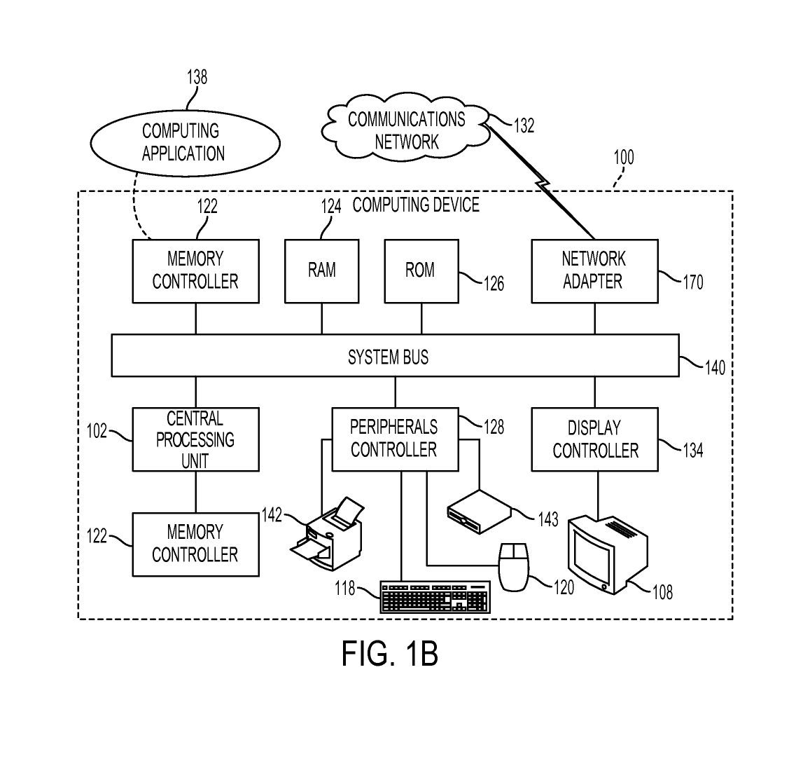

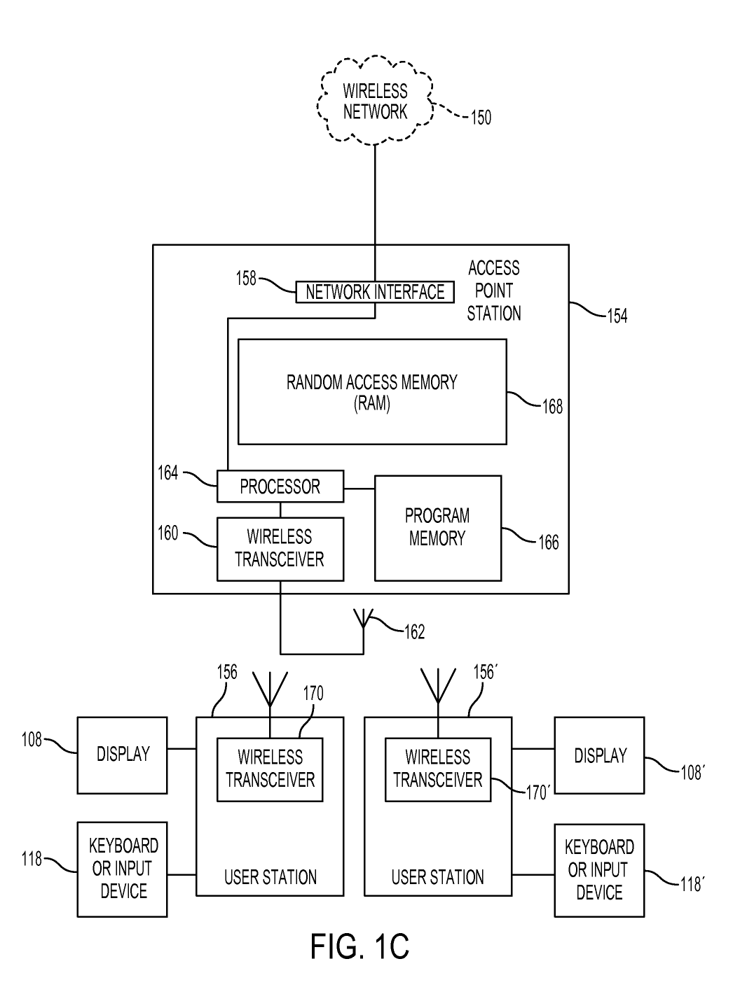

FIG. 1A is a block diagram showing a representative example logic device through which a browser can be accessed to implement the present invention. A computer system (or digital device) 100, which may be understood as a logic apparatus adapted and configured to read instructions from media 114 and/or network port 106, is connectable to a server 110, and has a fixed media 116. The computer system 100 can also be connected to the Internet or an intranet. The system includes central processing unit (CPU) 102, disk drives 104, optional input devices, illustrated as keyboard 118 and/or mouse 120 and optional monitor 108. Data communication can be achieved through, for example, communication medium 109 to a server 110 at a local or a remote location. The communication medium 109 can include any suitable means of transmitting and/or receiving data. For example, the communication medium can be a network connection, a wireless connection or an internet connection. It is envisioned that data relating to the present disclosure can be transmitted over such networks or connections. The computer system can be adapted to communicate with a participant and/or a device used by a participant. The computer system is adaptable to communicate with other computers over the Internet, or with computers via a server.

FIG. 1B depicts another exemplary computing system 100. The computing system 100 is capable of executing a variety of computing applications 138, including computing applications, a computing applet, a computing program, or other instructions for operating on computing system 100 to perform at least one function, operation, and/or procedure. Computing system 100 is controllable by computer readable storage media for tangibly storing computer readable instructions, which may be in the form of software. The computer readable storage media adapted to tangibly store computer readable instructions can contain instructions for computing system 100 for storing and accessing the computer readable storage media to read the instructions stored thereon themselves. Such software may be executed within CPU 102 to cause the computing system 100 to perform desired functions. In many known computer servers, workstations and personal computers CPU 102 is implemented by micro-electronic chips CPUs called microprocessors. Optionally, a co-processor, distinct from the main CPU 102, can be provided that performs additional functions or assists the CPU 102. The CPU 102 may be connected to co-processor through an interconnect. One common type of coprocessor is the floating-point coprocessor, also called a numeric or math coprocessor, which is designed to perform numeric calculations faster and better than the general-purpose CPU 102.

As will be appreciated by those skilled in the art, a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Some embodiments may be implemented in one or a combination of hardware, firmware and software. Embodiments may also be implemented as instructions stored on a non-transitory computer-readable storage medium, which may be read and executed by at least one processor to perform the operations described herein. A non-transitory computer-readable storage medium may include any mechanism for storing information in a form readable by a machine (e.g., a computer). For example, a non-transitory computer-readable storage medium may include read-only memory (ROM), random-access memory (RAM), magnetic disk storage media, optical storage media, flash-memory devices, and other non-transitory media.

In operation, the CPU 102 fetches, decodes, and executes instructions, and transfers information to and from other resources via the computer's main data-transfer path, system bus 140. Such a system bus connects the components in the computing system 100 and defines the medium for data exchange. Memory devices coupled to the system bus 140 include random access memory (RAM) 124 and read only memory (ROM) 126. Such memories include circuitry that allows information to be stored and retrieved. The ROMs 126 generally contain stored data that cannot be modified. Data stored in the RAM 124 can be read or changed by CPU 102 or other hardware devices. Access to the RAM 124 and/or ROM 126 may be controlled by memory controller 122. The memory controller 122 may provide an address translation function that translates virtual addresses into physical addresses as instructions are executed.

In addition, the computing system 100 can contain peripherals controller 128 responsible for communicating instructions from the CPU 102 to peripherals, such as, printer 142, keyboard 118, mouse 120, and data storage drive 143. Display 108, which is controlled by a display controller 163, is used to display visual output generated by the computing system 100. Such visual output may include text, graphics, animated graphics, and video. The display controller 134 includes electronic components required to generate a video signal that is sent to display 108. Further, the computing system 100 can contain network adaptor 136 which may be used to connect the computing system 100 to an external communications network 132.

### II. Networks and Internet Protocol

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

### III. Wireless Networks

Wireless networks can incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

In addition, Multiple Interface Devices (MIDs) may be utilized in some wireless networks. MIDs may contain two independent network interfaces, such as a Bluetooth interface and an 802.11 interface, thus allowing the MID to participate on two separate networks as well as to interface with Bluetooth devices. The MID may have an IP address and a common IP (network) name associated with the IP address.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi (Wireless Fidelity) devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

Wireless networks can also involve methods and protocols found in, Mobile IP (Internet Protocol) systems, in PCS systems, and in other mobile network systems. With respect to Mobile IP, this involves a standard communications protocol created by the Internet Engineering Task Force (IETF). With Mobile IP, mobile device users can move across networks while maintaining their IP Address assigned once. See Request for Comments (RFC) 3344. NB: RFCs are formal documents of the Internet Engineering Task Force (IETF). Mobile IP enhances Internet Protocol (IP) and adds a mechanism to forward Internet traffic to mobile devices when connecting outside their home network. Mobile IP assigns each mobile node a home address on its home network and a care-of-address (CoA) that identifies the current location of the device within a network and its subnets. When a device is moved to a different network, it receives a new care-of address. A mobility agent on the home network can associate each home address with its care-of address. The mobile node can send the home agent a binding update each time it changes its care-of address using Internet Control Message Protocol (ICMP).

FIG. 1C depicts components that can be employed in system configurations enabling the systems and technical effect of this disclosure, including wireless access points to which client devices communicate. In this regard, FIG. 1C shows a wireless network 150 connected to a wireless local area network (WLAN) 152. The WLAN 152 includes an access point (AP) 154 and a number of user stations 156, 156′. For example, the network 150 can include the Internet or a corporate data processing network. The access point 154 can be a wireless router, and the user stations 156, 156′ can be portable computers, personal desk-top computers, PDAs, portable voice-over-IP telephones and/or other devices. The access point 154 has a network interface 158 linked to the network 150, and a wireless transceiver in communication with the user stations 156, 156′. For example, the wireless transceiver 160 can include an antenna 162 for radio or microwave frequency communication with the user stations 156, 156′. The access point 154 also has a processor 164, a program memory 166, and a random access memory 168. The user station 156 has a wireless transceiver 170 including an antenna 172 for communication with the access point station 154. In a similar fashion, the user station 156′ has a wireless transceiver 170′ and an antenna 172 for communication to the access point 154. By way of example, in some embodiments an authenticator could be employed within such an access point (AP) and/or a supplicant or peer could be employed within a mobile node or user station. Desktop 108 and key board 118 or input devices can also be provided with the user status.

### IV. Computer Network Environment

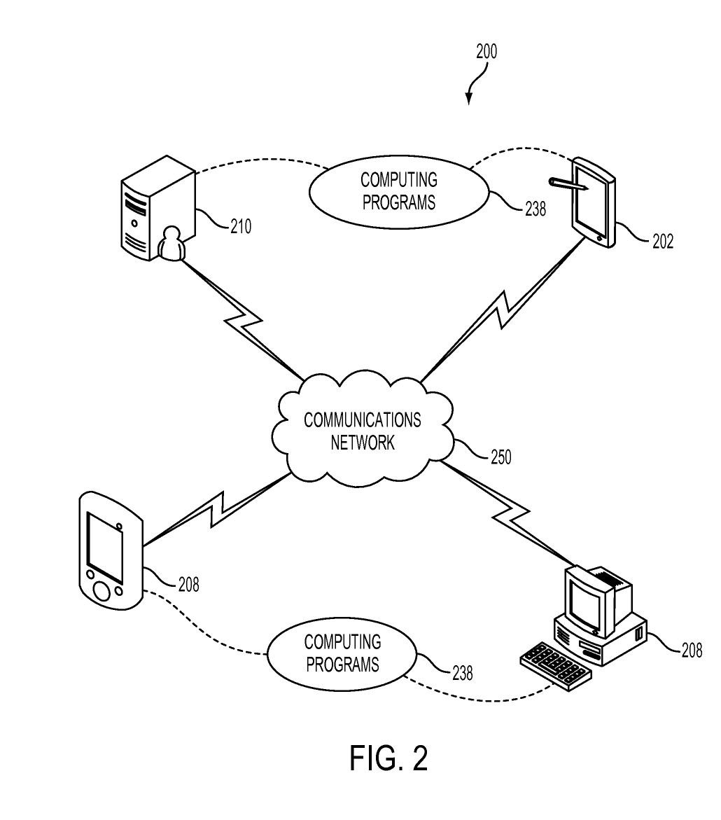

Computing system 100, described above, can be deployed as part of a computer network used to achieve the desired technical effect and transformation. In general, the above description for computing environments applies to both server computers and client computers deployed in a network environment. FIG. 2 illustrates an exemplary illustrative networked computing environment 200, with a server in communication with client computers via a communications network 250. As shown in FIG. 2, server 210 may be interconnected via a communications network 250 (which may be either of, or a combination of a fixed-wire or wireless LAN, WAN, intranet, extranet, peer-to-peer network, virtual private network, the Internet, or other communications network) with a number of client computing environments such as tablet personal computer 202, smart phone 208, personal computer 202, and personal digital assistant. In a network environment in which the communications network 250 is the Internet, for example, server 210 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment 200 can utilize various data security protocols such as secured socket layer (SSL) or pretty good privacy (PGP). Each client computing environment can be equipped with operating system 238 operable to support one or more computing applications, such as a web browser (not shown), or other graphical user interface (not shown), or a mobile desktop environment (not shown) to gain access to server computing environment 200.

In operation, a user (not shown) may interact with a computing application running on a client computing environment to obtain desired data and/or computing applications. The data and/or computing applications may be stored on server computing environment 200 and communicated to cooperating users through client computing environments over exemplary communications network 250. The computing applications, described in more detail below, are used to achieve the desired technical effect and transformation set forth. A participating user may request access to specific data and applications housed in whole or in part on server computing environment 200. These data may be communicated between client computing environments and server computing environments for processing and storage. Server computing environment 200 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### V. Devices for Measuring Cough and Respiratory-Rate which are Configurable to Operate in the Computing and Network Environments to Achieve a Desired Technical Effect or Transformation

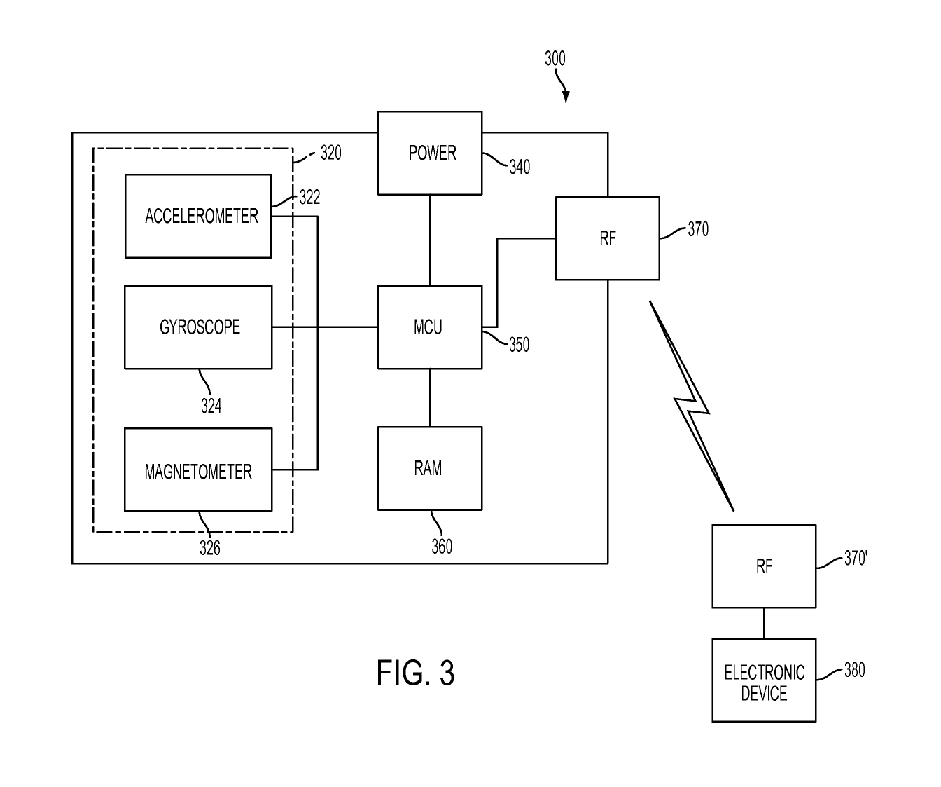

FIG. 3 illustrates a block diagram of a measurement device 300 according to the disclosure. The measurement device 300 has one or more sensors 320, such as an AGM sensor which includes an accelerometer 322, a gyroscope 324, and a magnetometer 326. The one or more sensors 320 are in communication with a microprocessor 350, which is in communication with a memory, such as random access memory 360. Additionally, the device may include RF 370 capability and a removeable power supply 340, such as a battery. The device 300 is in communication via RF 370′ with a secondary electronic device 380. Suitable secondary electronic devices include, for example, mobile phones, tablets, and computers. The AGM sensor is configurable to measure changes in velocity (directly, as the acceleration is the first time derivative of the velocity) and absolute orientation sensor in the UP-DOWN plane; changes in orientation (regular gyro or integrating rate gyro) or changes in rotational velocity (rate gyro); and absolute orientation in a plane, such as a NESW plane.

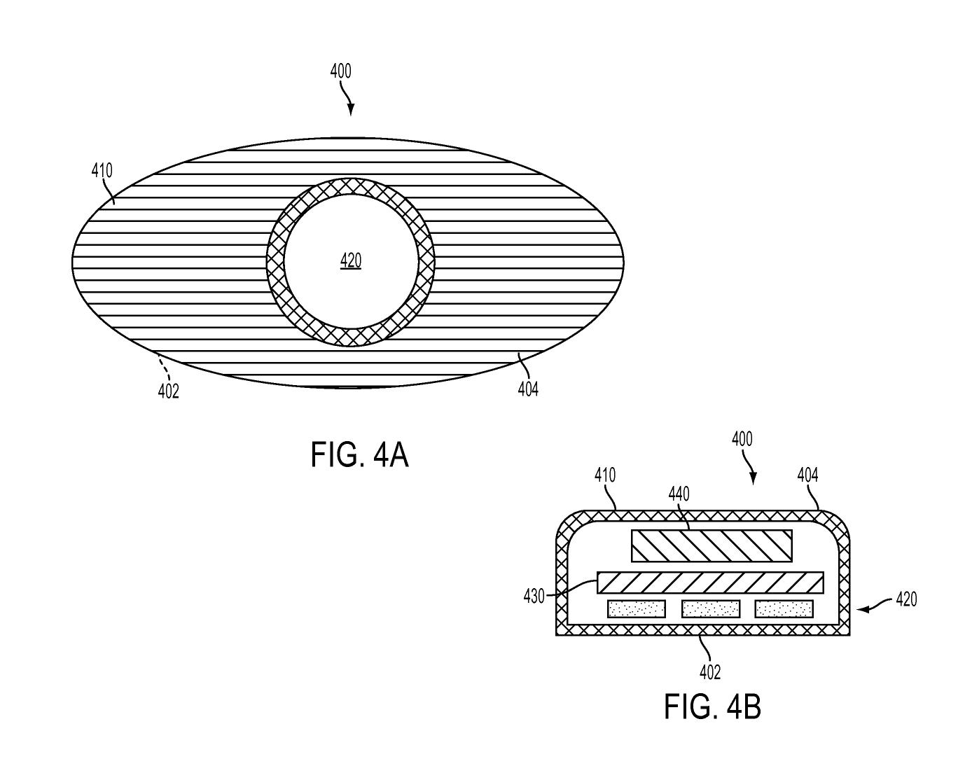

FIGS. 4A-B illustrate a bottom view and a side cross-sectional view of a measurement device 400 according to the disclosure. The measurement device 400 has an upper side 402 and a lower side 404 adapted to engage a mammalian body. The lower surface 404 also places one or more sensors 420 in proximity to the mammalian body when deployed. Turning to FIG. 4B the measurement device 400 can have a housing 410, such as a flexible housing, which houses one or more sensors 420, such as an accelerometer, a gyroscope, and/or a magnetometer. A power source 440, such as battery, is provided to power the device during operation. Additionally, a PCB 430 can be incorporated into the device housing 410.

Any measurement device 400 of the disclosure can be configured such that a layer of bioadhesive is provided to facilitate secure contact by the measurement device and the mammalian body. The layer can comprise any suitable bioadhesive, or film, nonwovens or meshes that are treated with bioadhesive. In another configuration a strap or band is provided that is configured to removeably secure one or more measurement devices to the patient. Closure mechanism include, but are not limited to, Velcro®, buckles, snaps, or any other suitable mechanism

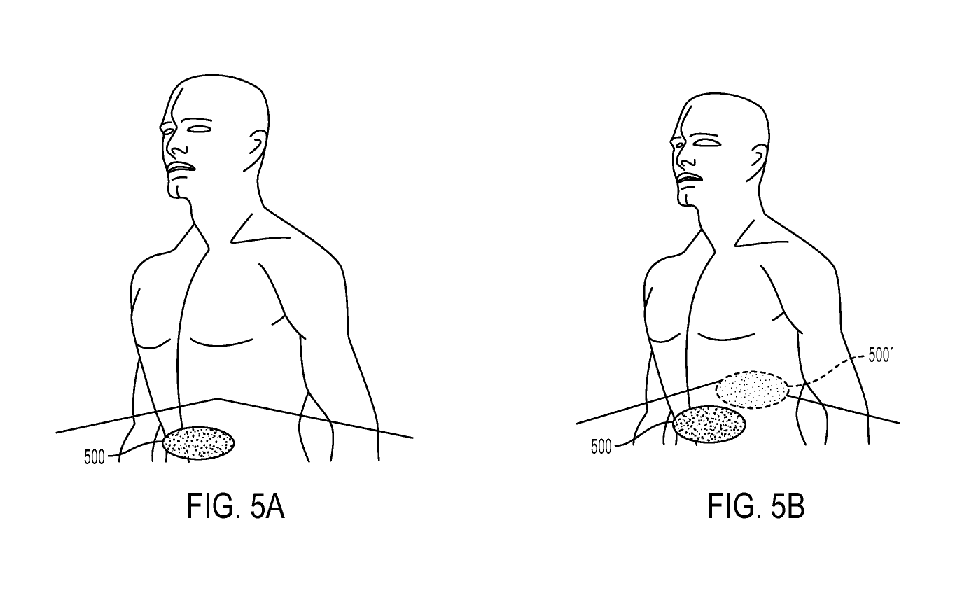

FIGS. 5A-B illustrate potential device placement locations on a torso of a mammal. For purposes of illustration, a human torso is illustrated. However, as will be appreciated by those skilled in the art, the device can be used for a variety of other mammals, including, but not limited to, horses, dogs, and cats. As shown in FIG. 5A, a single sensor is positioned ventrally.

As shown in FIG. 5B a first sensor 500 is positioned ventrally and a second sensor 500′ is positioned dorsally. In a two sensor configuration, a first sensor is compared to the second sensor to determine subject position and compare signals for noise and characteristics of cough and respiration. In some cases the first sensor could be used to capture a first set of measurements, and a second sensor is used to capture a second set of measurements, which are used as a control to assess position and noise that might exist in the first set of measurements thus providing relative position of the mammal and improving the signal and measurement of cough and respiration.

In the embodiment of FIG. 5B, the second sensor can, for example, be a second AGM sensor configured the same as the first AGM sensor.

In another configuration, the second sensor can be a piezoelectric phonopneumogragh vibratory (PPG) sensor or acoustic sensor that is securely positioned near the trachea. In still another configuration, the two sensor configuration of FIG. 5B is accompanied by a third acoustic sensor.

As the sensor information is processed, the system looks for distinctive shapes to identify a cough signature, which depending on the position of the mammal can exist in one of these respective directions (X, Y, or Z). In this example, Z provides the most compliant axis and best signal for measurement.

Other aspects include one or more networked devices. The networked devices comprise: a memory; a processor; a communicator; a display; and an apparatus for detecting expiry flow-rate as discussed herein.

In some aspects communication systems are provided. The communication systems comprise: an apparatus for detecting cough as described herein; a server computer system; a measurement module on the server computer system for permitting the transmission of a cough measurement from the device for measuring the characteristic of the cough over a network; at least one of an API engine connected to at least one of the system for measuring the characteristic of the cough to create a message about the cough measurement and transmit the message over an API integrated network to a recipient having a predetermined recipient user name, an SMS engine connected to at least one of the system for measuring the characteristic of the cough to create an SMS message about the cough measurement and transmit the SMS message over a network to a recipient device having a predetermined cough measurement recipient telephone number, and an email engine connected to at least one of the system for measuring the characteristic of the cough to create an email message about the cough measurement and transmit the email message over the network to a cough measurement recipient email having a predetermined cough measurement recipient email address. A storing module can also be provided on the server computer system for storing the cough measurement on the system for measuring the characteristic of the cough server database. Moreover, at least one of the system for measuring the characteristic of the cough is connectable to the server computer system over at least one of a mobile phone network and an Internet network, and a browser on the cough measurement recipient electronic device is used to retrieve an interface on the server computer system. Additionally, a plurality of email addresses are held in a system for measuring the characteristic of the cough database and fewer than all the email addresses are individually selectable from the computer system, the email message being transmitted to at least one cough measurement recipient email having at least one selected email address. In some instances at least one of the system for measuring the characteristic of the cough is connectable to the server computer system over the Internet, and a browser on the cough measurement recipient electronic device is used to retrieve an interface on the server computer system. Where the system is in communication with, for example, a healthcare provider a plurality of user names are held in the system for detecting coughs database and fewer than all the user names are individually selectable from the computer system, the message being transmitted to at least one cough measurement recipient user name via an API. The cough measurement recipient electronic device can also be connectable to the server computer system over the Internet, and a browser on the cough measurement recipient electronic device is used to retrieve an interface on the server computer system. The cough measurement recipient electronic device may also be connected to the server computer system over a cellular phone network, such as where the electronic device is a mobile device. Additionally, the system can include an interface on the server computer system, the interface being retrievable by an application on the cough measurement recipient mobile device. In some cases, the SMS cough measurement is received by a message application on the cough measurement recipient mobile device. Where a plurality of SMS cough measurements are received for the cough measurement, each by a respective message application on a respective cough measurement recipient mobile device. At least one SMS engine can be configured to receive an SMS response over the cellular phone SMS network from the cough measurement recipient mobile device and stores an SMS response on the server computer system. Additionally, a cough measurement recipient phone number ID is transmitted with the SMS cough measurement to the SMS engine and is used by the server computer system to associate the SMS cough measurement with the SMS response. Moreover, the server computer system can be connectable over a cellular phone network to receive a response from the cough measurement recipient mobile device. The SMS cough measurement can also include a URL that is selectable at the cough measurement recipient mobile device to respond from the cough measurement recipient mobile device to the server computer system, the server computer system utilizing the URL to associate the response with the SMS cough measurement. The communication system can further comprise in at least some configurations: a downloadable application residing on the cough measurement recipient mobile device, the downloadable application transmitting the response and a cough measurement recipient phone number ID over the cellular phone network to the server computer system, the server computer system utilizing the cough measurement recipient phone number ID to associate the response with the SMS cough measurement. In other configurations, the system can comprise: a transmissions module that transmits the cough measurement over a network other than the cellular phone SMS network to a cough measurement recipient user computer system, in parallel with the cough measurement that is sent over the cellular phone SMS network, and/or a downloadable application residing on the cough measurement recipient host computer, the downloadable application transmitting a response and a cough measurement recipient phone number ID over the cellular phone network to the server computer system, the server computer system utilizing the cough measurement recipient phone number ID to associate the response with the SMS cough measurement.

An additional cough detector or sensor can be used in conjunction with the system to provide an additional mechanism to identify and confirm coughs. Such sensor would be configurable to draw a distinction between coughing sounds and other sounds, such as non-cough noises. The sensor is also configurable such that it is also capable of differentiating between cough-induced vibration or sounds of tracheal air and other rapid movements. A PPG or Microphone sensor placed over the trachea at the anterior cervical area is used to collect the sound or vibratory signals. The sensor collects the signal and transduces it into an electric signal. Any sensor known in the art able to detect the acoustic signal of cough and the frequency signal generated by the associated rapid flow of air and tracheal deformation during cough is applicable. The detection of frequencies associated with cough may be achieved using a single sensing element in which case the sensor is simple.

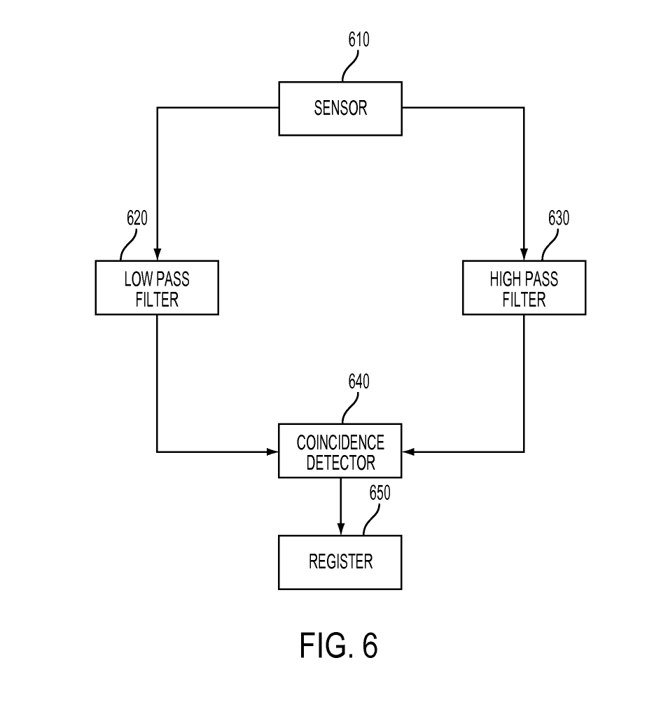

An example of a second or third sensor for use in the system is-shown schematically in FIG. 6. The electrical output of the sensor 610 is channeled to two separate frequency filters 620 and 630. Low pass filter 620 selectively passes signals associated with events that correspond to the rapid expiratory flow of air in the trachea or the sudden change in the tracheal dimensions associated with its dynamic collapse during cough. Such events typically generate vibrations or sound in the frequency range of 0.5 to 10 Hz. High pass filter 630 selectively passes signal associated with brief loud sounds in the frequency range of 200 to 2000 Hz. The amplitude threshold is typically 70 dB absolute sound level pressure or higher and the duration threshold is typically between 0.15 to 1.5 sec or narrower. Coincidence detector 640 receives signals from both filters 620, 630. In the event that signals from both filters arrive simultaneously, coincidence detector 640 analyzes the received signals and identifies the event as a coughing event or determines that the signal is not a coughing event. This data is then transmitted to a data register 650. The data register keeps log of the coughing activity detected by sensor 610. Data from the register is compared to data from the AGM sensor(s) to determine the agreement of the presence of a cough and mark it for counting. Additional secondary sensors include, for example, sensors described in U.S. Pat. No. 8,241,223 B2 issued Aug. 14, 2012, to Gavriely et al. for Cough Detector.

The operation of the ultrasonic tracheal cough detector is described. Oscillator generates an ultrasound frequency energy that drives the ultrasonic emitter. The returning signal is picked up by the ultrasonic receiver and amplified by the amplifier. The frequency shift detector receives the signals from the amplifier and through a secondary channel the signal from the oscillator. The frequency shift detector determines if there was a frequency shift and if this frequency shift exceeded a threshold. If the threshold was exceeded as indicated by the yes over the arrow, a signal is transmitted from the frequency shift detector to the duration detector. In an alternative embodiment, the coincidence between the signal indicating that the frequency shift threshold was exceeded and the high frequency signal from the microphone built into the sensor (not shown) is evaluated. Only if there is a coincidence between the timing of both signals the duration detector receives a signal. The duration detector determines if the duration of the signal it receives is within the time duration of a cough as described above and if so, generates a signal that triggers the data logging element to register the event as the presence of a cough. In addition, the time of the event, determined by the timer is recorded in the data logger. Optionally, the raw data from the preceding time period of 2-3 seconds, transiently stored in the raw data recorder may be recorded in the data logger for subsequent analysis or validation and agreement with the AGM Torso sensor(s). The data stored in the data logger may be transmitted via a transmission element to a permanent storage and analysis element via wire or wireless transmission as a digital or an analog signal. The data from the analyzer may be displayed in graphic or numeric form by the data display element 766.

### VI. Kits

Bundling all devices, tools, components, materials, and accessories needed to use a device to test cough and respiration into a kit may enhance the usability and convenience of the devices. Suitable kits include one or more AGM sensors, and one or more acoustic sensors. Suitable kits, can also include, for example, a securement mechanism (e.g., strap or belt or adhesive pad), a respiration flow measurement device, power supplies, software programs (apps) configurable to collect information from the devices and/or provide information to a central database or system, alcohol swabs, and the like.

### VII. Examples

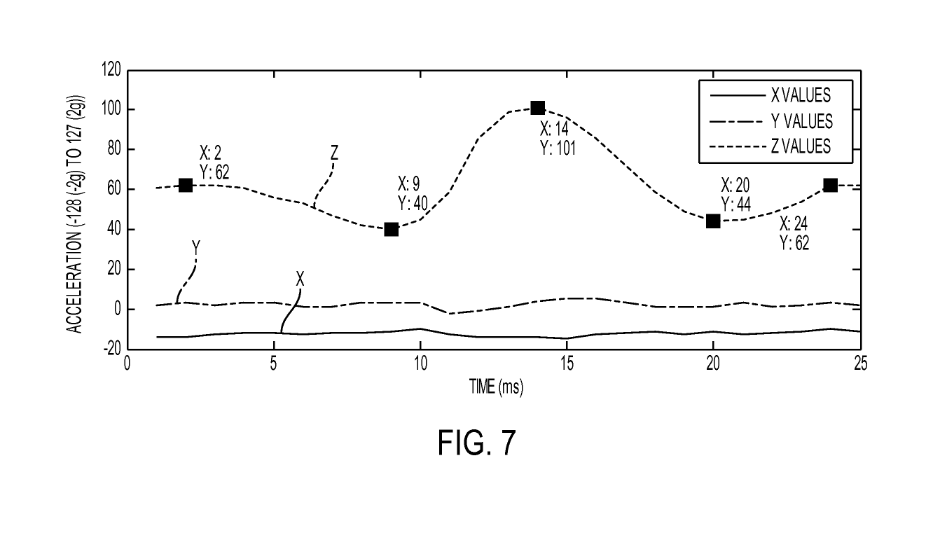

FIG. 7 illustrates cough output for a patient lying prone on a bed taken using a single sensor having X values, Y values and Z values. In this example, the sensor measurement reflects the actual measurement taken by a single sensor without comparison to a control sensor. In this optimal condition a single sensor is capable of generating an acceptable signal. It is unlikely that a subject could maintain this position throughout the entire period of measurement. In this position, the Z axis provides adequate amplitude to allow detection of cough.

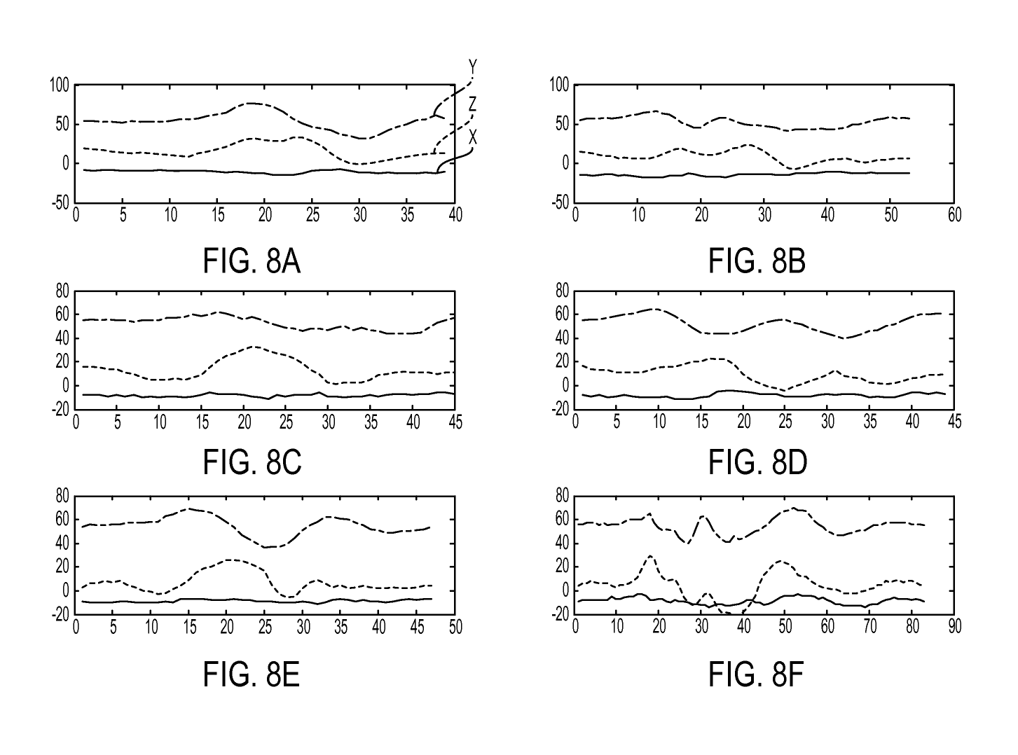

FIGS. 8A-F illustrate cough outputs for various subjects in semi recumbent positions with different outputs among the axis of the AGM sensors attached to the patient. As illustrated, various axis can produce higher amplitudes and in some cases (FIGS. 8A-C) with varying amplitude agreement in the cyclic waveform is seen. In cases when a device is affixed to the body using a tight belt or adhesive (FIGS. 8D-F) and measurements are taken using a single sensor having X values, Y values and Z values improved cyclical agreement and measurement of a cough is made. In this example, the sensor is an AGM. The measurement reflects the actual measurement taken by a single sensor without comparison to a control sensor.

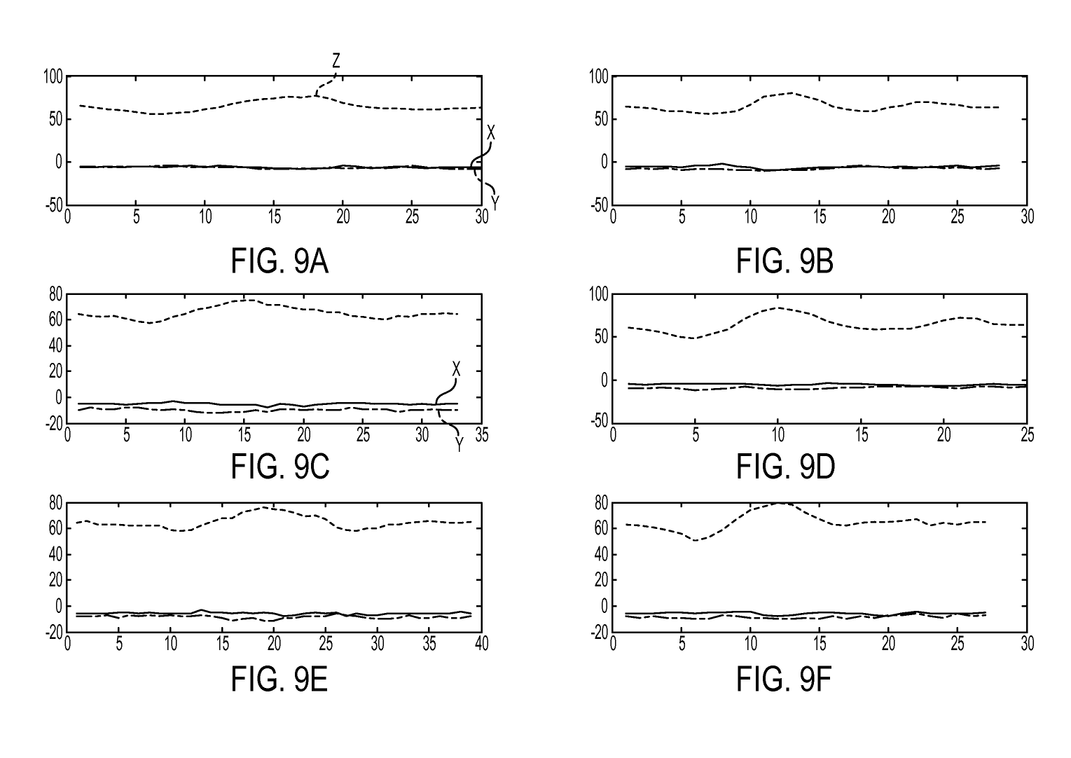

FIGS. 9A-F illustrate another example of cough output for a patient when the patient is lying prone on a bed with sensor affixed optimally using a belt and taken using a single sensor having X values, Y values and Z values. In this example, the sensor is an AGM. The measurement reflects the actual measurement taken by a single accelerator output of a sensor without comparison to a control sensor. In this example milder less amplitude and cyclical waves are observed illustrating that different subjects cough with varying amplitude and rhythm, thus increasing the importance of agreement with cross-correlation of a secondary AGM or Tracheal Sensor.

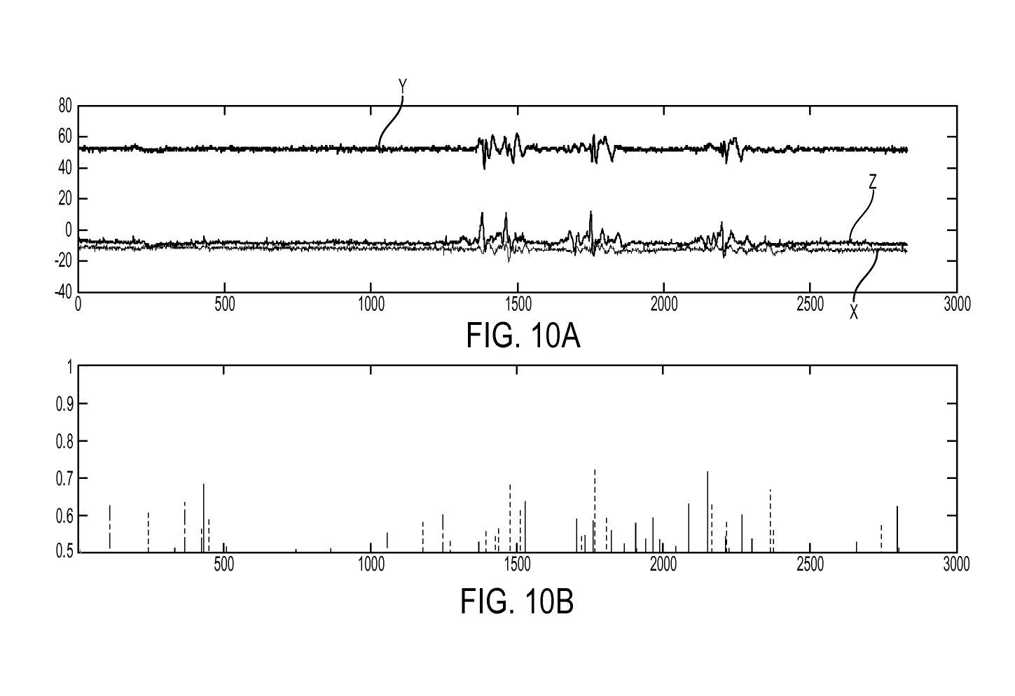

FIGS. 10A-B illustrate a cross-correlation utilizing a second AGM appropriately conditioned and digitized. In this illustration multiple coughs, which normally are seen as arrhythmic, are presented and filtered using a proprietary cough profile to produce a measurable determination of coughing events in the illustration below.

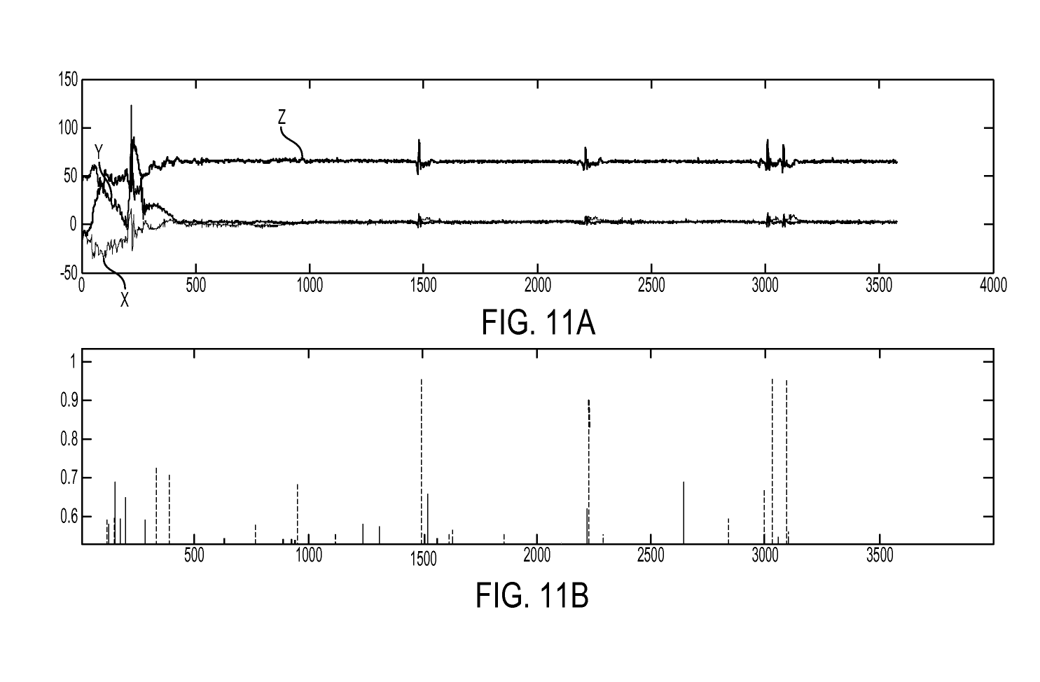

FIGS. 11A-B illustrate a cross-correlation utilizing a second AGM with a subject in motion and after 500 msec appropriately conditioned, digitized and cross correlated to produce a measurable threshold for detecting and counting a single cough.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A cough detector comprising:
a housing adaptable to engage an abdomen or thorax of a mammal;
a first sensor comprising an AGM sensor positioned within the housing;
a processor in communication with the AGM sensor wherein the processor is capable of receiving a signal from the AGM sensor and analyzing the signal to determine whether the signal is characterizable as a cough; and
a power source.

2. The detector of claim 1 wherein the processor is positioned within the housing.

3. The detector of claim 1 wherein the processor is in wireless or wired communication with the sensor in the housing.

4. The detector of claim 1 wherein the first sensor is capable of measuring one or more of each of changes in velocity, movement, orientation of the sensor in a plane, and changes in rotational velocity.

5. The detector of claim 1 wherein the first sensor is capable of determining one or more of a power of a movement resulting from the cough, a motion resulting from the cough, and a change in orientation resulting from the cough.

6. The detector of claim 1 wherein data relating to the cough is transmitted to a remote host computer or a data logger via a transmitter.

7. The detector of claim 1 further comprising a securement device capable of securely positioning the sensor adjacent an abdomen or thorax of a patient.

8. The detector of claim 1 further comprising:
one or more secondary sensors, wherein the one or more secondary sensors are selected from the group comprising an AGM sensor, a PPG sensor, and a contact sensor capable of simultaneously sensing and transducing a range of low frequency to high frequency mechanical vibrations, wherein the sensor is configured to be positioned and brought into contact over a lower segment of a cervical trachea of the mammal.

9. The detector of claim 8 wherein the one or more secondary sensors are in wireless or wired communication with the first sensor.

10. The detector of claim 8 wherein the processor in communication with the first sensor is in communication with the one or more secondary sensors.

11. The detector of claim 8 wherein the processor is capable of comparing one or more signals from the first sensor to one or more signals from the one or more secondary sensors to determine the presence of a cough.

12. The detector of claim 8 further comprising one or more processors in communication with the each of the one or more secondary sensors wherein the one or more processors are capable of receiving a signal from the one or more secondary sensors.

13. The detector of claim 12 wherein the one or more processors are capable of comparing one or more signals from the first sensor to one or more signals from the one or more secondary sensors to determine the presence of a cough.

14. The detector of claim 8 further comprising
a coincidence detector for receiving signals from one or more of the first sensor and the one or more secondary sensors, wherein the coincidence detector identifies a coincidence between the signals arriving from two of the sensors and generates a continuous signal for the duration of the coincidence;
a duration detector for determining whether the duration of the continuous signal generated by the coincidence detector is within a respective characterization of cough, to thereby identify coughing events; and
a register for storing data concerning coughing events identified as such by the duration detector.

15. The detector of claim 8 further comprising one or more secondary housings for the one or more secondary sensors wherein at least one of the one or more secondary housings is adaptable to engage a mammal either above or below a diagram in opposition to the first sensor.

16. The detector of claim 8 further comprising one or more secondary housings for the one or more secondary sensors wherein at least one of the one or more secondary housings is adaptable to contact over a lower segment of a cervical trachea of the mammal

17. The detector of claim 8 further comprising one or more secondary power sources capable of providing power to the one or more secondary sensors.

18. The detector of claim 8 wherein at least one of the one or more secondary sensors is capable of measuring a frequency range of 20 Hz to 2000 Hz.

19. The detector of claim 8 wherein at least one of the one or more secondary sensors is capable of acoustic sensing using one or more of a vibration sensor and an audible sensor.

20. A method for detecting and counting coughing events comprising:
a) positioning a cough detector comprising a housing, a first sensor comprising an AGM sensor, a power source, and a processor in communication with the AGM sensor on an abdomen or thorax of a mammal;
b) detecting one or more of orientation, motion and pressure;
c) analyzing the one or more of orientation, motion and pressure sensed to determine whether the detected orientation, motion and pressure corresponds to a movement, motion or pressure resulting from a cough.

21. The method for detecting and counting coughing events according to claim 20 further comprising the step of transmitting the detected orientation, motion and pressure to a remote host computer or a data logger via a wired or wireless transmitter.

22. The method for detecting and counting coughing events according to claim 20 further comprising the steps of:
a) positioning one or more of a secondary sensor over the abdomen or thorax in opposition to the first AGM sensor or over a lower segment of a cervical trachea for simultaneously sensing and transducing mechanical vibrations emanating from the cervical trachea segment;
b) identifying, by a coincidence detector, coincidence of signals;
c) determining, by a duration detector, whether the duration of the coincidence is within a range of cough durations to thereby identify coughing events; and
d) storing, by a register, data concerning coughing events identified as such by the duration detector.

23. The method for detecting and counting coughing events according to 20 further comprising the step of sensing one or more of a tracheal vibration or an acoustic signal within a frequency range of 20 Hz to 2000 Hz.

24. The method for detecting and counting coughing events according to claim 20 further comprising the step of: comparing one or more signals from the first sensor to one or more signals from the second sensor to determine the presence of a cough.

25. The method for detecting and counting coughing events according to claim 20 further comprising the steps of: removably securing the first sensor to the abdomen or thorax of the mammal.

26. The method for detecting and counting coughing events according to claim 22 further comprising the steps of: removably securing the one or more of secondary sensors to one or more of the abdomen, thorax, or trachea of the mammal.

27. A system comprising:
a cough detector configurable to be in communication with a communication network, wherein the cough detector comprises a housing adaptable to engage an abdomen or thorax of a mammal, a first sensor comprising an AGM sensor positioned within the housing; and
a processor in communication with the AGM sensor wherein the processor is capable of receiving a signal from the AGM sensor and analyzing the signal to determine whether the signal is characterizable as a cough, and a power source;
an alert generator.

28. The system of claim 27 wherein the alert generator delivers an alert over the communication network.

29. The system of claim 27 wherein the alert is an alert to advise a healthcare provider of a physiological condition as a result of the cough detection.

30. The system of claim 27 further comprising a report generator.

31. The system of claim 30 wherein the report generator compiles information received from the sensor and generates a report.
